(19) United States
(12) Patent Application Publication
(10) Pub. No.: US 2007/0240488 A1
(43) Pub. Date: Oct. 18, 2007

(54) Molten Metal Gas Sampling

(76) Inventors: Joseph J Kreuser, Champlin, MN (US); Thomas W. Junker, Shorewood, WI (US); Richard A. Falk, Holmes Beach, FL (US)

Correspondence Address: FOSTER, SWIFT, COLLINS & SMITH, P.C., 313 SOUTH WASHINGTON SQUARE, LANSING, MI, 48933, US

(21) Appl. No.: 11/660,484

(22) PCT Filed: Sep. 14, 2005
(86) PCT No.: PCT/US05/32775

§ 371 Date: Feb. 16, 2007

Related U.S. Application Data
(60) Provisional application No. 60/522,317, filed on Sep. 14, 2004.

(51) Int. Cl. G01N 33/20

(52) U.S. Cl. 73/19.07

(57) ABSTRACT

Apparatus and methods for simultaneously testing molten metal, such as steel, for concentration of three or more dissolved gases. A gas flow manifold allows multiple calibrations of a gas analysis device independently of operations such as immersion and purging of an immersible probe that can be provided with a bypass conduit for gas flow directly to the analysis device. Accurate nitrogen content is determined by oxidizing a retrieved gas sample using heated CuO and measuring gas concentration of the gas before and after such oxidation. A gas reservoir can be used to temporarily store a portion of the retrieved gas for use in such testing.

### FIELD OF THE INVENTION

The invention relates to apparatus and methods for measurement of gas concentrations in molten metal. More particularly the invention relates to apparatus and methods to determine the quantity of dissolved gases and, preferably, the simultaneous measurement of several gases such as hydrogen, oxygen, or nitrogen in molten metals such as steel.

### BACKGROUND OF THE INVENTION

Various apparatus and processes have heretofore been used to measure the content of dissolved gases such as hydrogen, oxygen and nitrogen in molten metals such as molten aluminum or steel. Such devices and methods are disclosed in commonly owned U.S. Pat. No. 6,216,526, the disclosure of which is incorporated herein by reference. A continuing goal in the development of such apparatus has been to improve the speed and accuracy of such testing.

### SUMMARY OF THE INVENTION

The present invention relates to improvements over the equipment and methods described in earlier commonly owned U.S. Pat. No. 6,216,526. In accordance with an important aspect of the invention the system utilizes a gas analysis device such as a high speed mass spectrometer system which provides a response time for reading of contents of gases within a metallic melt of under two seconds with an accuracy of approximately one part in ten million. In accordance with the related aspect, the system can be utilized for measurement of gases contained in any molten metal or alloy melt at a temperature less than about 1704.44 degrees Celsius (3100 degrees Fahrenheit). An important feature of the present invention is the provision in the system of a manifold valve assembly that enables switching among several calibration gases without loss of accuracy of the resultant calibration results. A related advantage is the ability to perform calibration of the gas analysis device simultaneously with other operations such as purging of the associated immersible probe that is connected to the gas analysis device by means of a fluid flow conduit which bypasses the manifold.

Another aspect of the present invention is the utilization of such a manifold valve system that eliminates dead volume areas while providing flow changes among as many as six to ten or more valves. In accordance with another related aspect, the valves enable the rapid switching among different gases utilized for standardization or calibration of the system. In accordance with another related aspect, the flow and analysis of gases are controlled using a microprocessor to accurately and rapidly determine concentrations of measured gases within the system.

In accordance with another embodiment of the invention, a temporary storage reservoir is utilized in combination with the valves and a vacuum pump. In accordance with a related aspect, a gas stream retrieved from the molten metal is diverted into the reservoir when a steady state is achieved. The reservoir then allows continuing analysis of gases, enabling the system to simultaneously perform other functions. A portion of the gas stream may be analyzed while a second portion, retrieved from the reservoir, may be analyzed later, after subjecting it to a treatment, for example, oxidation. In accordance with another related aspect, a method is provided wherein the reservoir is emptied during each purge sequence and then sealed utilizing a valve such as a solenoid activated valve to make the reservoir, and hence the system, ready for measurement of the next sample.

In accordance with yet another embodiment of the invention, an oxidation furnace utilizing CuO is provided to convert any CO in the system to CO₂. Additionally, the furnace converts H₂ in the system to H₂O. In accordance with a related aspect, a process is provided wherein measurement of the gases obtained directly from the metallic melt is performed followed by a second measurement of the gases obtained subsequent to passage of the gases through the copper oxide furnace. This process, then, by eliminating carbon monoxide from the retrieved gases enables an accurate reading of nitrogen, which, like CO, has an atomic mass unit of 28.

In accordance with a further related aspect of the invention, a desiccant, which contains calcium sulfate or other hydroscopic material, is placed in line downstream or subsequent to the copper oxide furnace to prevent introduction of water into the mass spectrometer. In accordance with another related aspect of the invention, the decrease in mass unit 28 readings between the two measurements provides an accurate determination of the concentration of carbon monoxide contained in the molten metal.

In one embodiment, the invention provides method for determination of the concentration of gases dissolved in molten metal that includes providing a molten metal immersible probe, a gas analysis device, and a gas flow conduit interconnecting said probe and the gas analysis device, immersing the probe in a molten metal, introducing a stream of inert carrier gas into the metal through the probe, recovering the carrier gas as said gas bubbles out of said metal, using the analysis device to determine the content of at least one gas contained in the gas flowing out of the metal, passing a second amount of the carrier gas recovered from the metal through an oxidizing medium, thereby converting carbon monoxide contained in said gas to carbon dioxide and converting hydrogen contained in said gas to water vapor. The gas is then tested after passage thereof through said oxidizing medium, and the readings obtained from analysis of said two gas streams compared to thereby accurately determine the concentration of nitrogen, hydrogen and carbon monoxide contained in the gas. The oxidizing medium may consist of copper oxide heated in a copper oxide furnace.

In accordance with a further aspect of this embodiment, the second amount of the gas stream is temporarily stored in a reservoir prior to analysis thereof. The gas stream passing out of the oxidizing medium may be passed through a desiccant material to remove water vapor therefrom.

### BRIEF DESCRIPTION OF THE DRAWINGS

FIG. 1 is a schematic view illustrating an embodiment of the invention;

FIG. 2 is a fragmentary side view with interior parts shown by phantom lines of a manifold valve assembly used in practice of the invention;

FIG. 3 is a sectional view taken along Line 3-3 of FIG. 2;

FIG. 4 is a schematic view illustrating another embodiment of the invention;

FIG. 5 is a perspective view of a gas reservoir bag used in the practice of the invention;

FIG. 6 is a perspective view of a CuO furnace used in the practice of the invention; and

FIG. 7 is a partial sectional view of a treatment chamber of the furnace shown in FIG. 6.

### DETAILED DESCRIPTION OF THE INVENTION

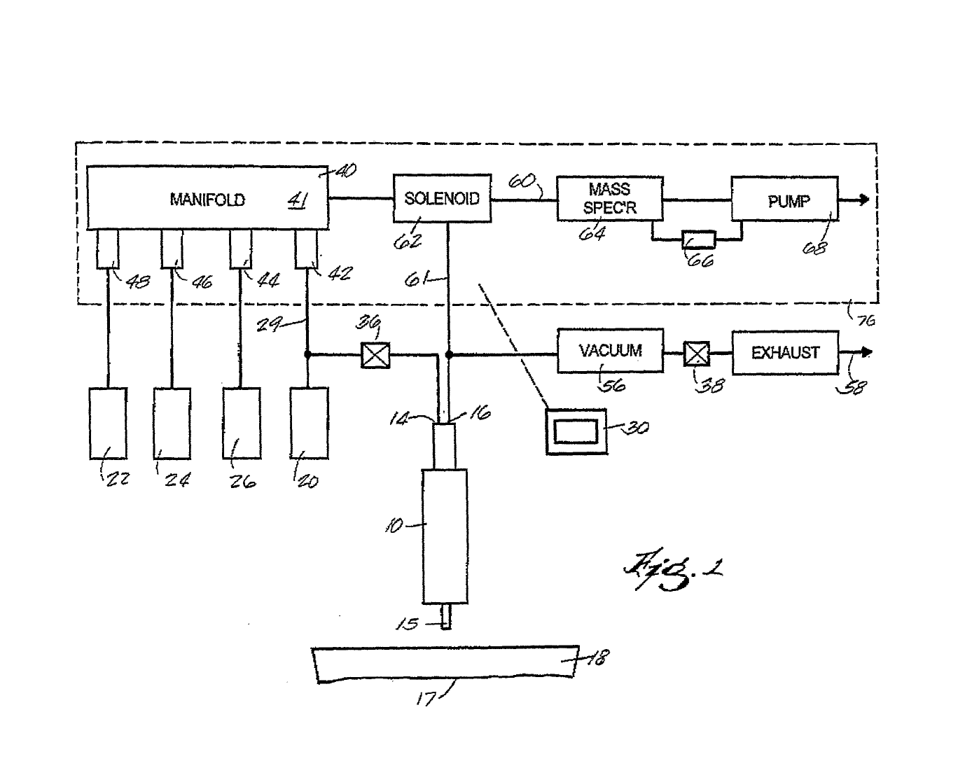

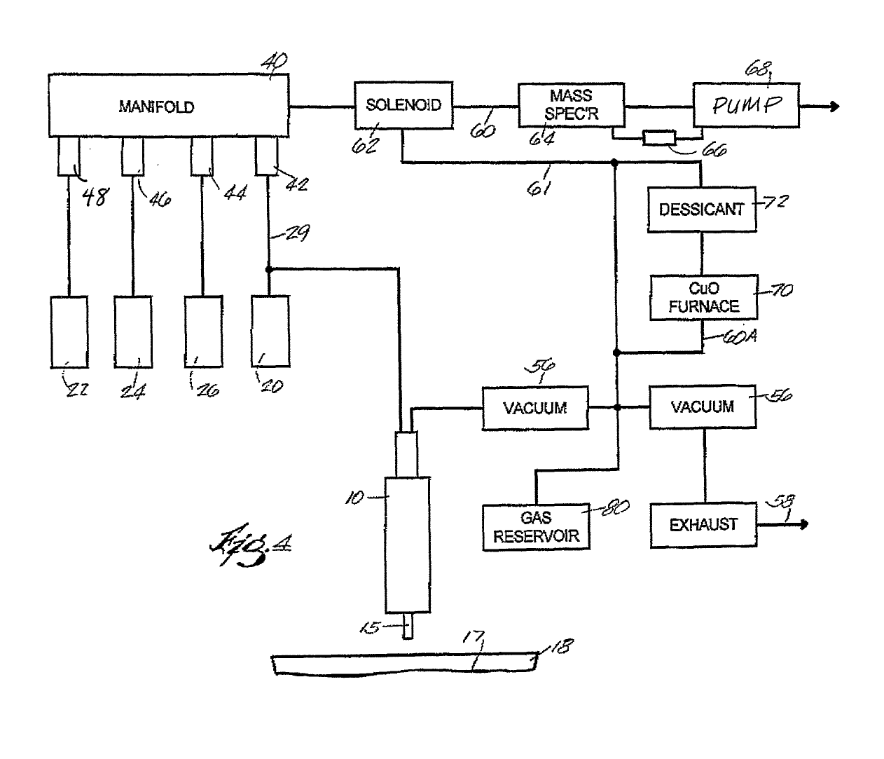

Referring to FIG. 1, a schematic view of one embodiment of the invention includes a probe assembly 10 similar to that described in detail with reference to FIG. 4 of U.S. Pat. No. 6,216,526. Probe 10 is immersible into a metal bath 17 (contained in a vessel 18) to determine the content of gases dissolved therein. The concentration of hydrogen, oxygen, and nitrogen dissolved in a molten iron or steel bath can be determined simultaneously. Additionally, the amount of carbon monoxide can be determined.

In this embodiment several sources of gas, a carrier gas 20, preferably argon, and three calibration gases of known composition 22, 24, and 26 are utilized. Solenoid activated valves 42, 44, 46 and 48, respectively, control flow from pressurized tanks providing the gases 20, 26, 24, and 22, respectively. Additional solenoid activated valves 36 and 38, are provided in the flow lines as will be further explained hereinafter.

The lines from pressurized gas sources 20, 22, 24, and 26 lead via gas flow conduits to a valve manifold assembly 40. Valve manifold assembly 40 includes a block 41 that is provided with valve ports electronically controlled by solenoid actuated valves 42, 44, 46 and 48. While four ports are shown for purposes of illustration, it will be understood that a larger number, for example 8 to 10, may be used as needed for the requirements of a particular application. A mass spectrometer inlet capillary tube 60 leads to a quadrupole mass spectrometer 64 that additionally is connected to turbo pump 66 and a rotary vane, membrane, or scrolling pump 68.

A remote computer 30 is electronically connected to the mass spectrometer 64 and associated valve manifold assembly 40 as well as the associated pumps. A vortex cooler (not shown) preferably using compressed air or another gas as a coolant is provided to cool the interior of an enclosure 76 within which the mass spectrometer 64 and associated controls are contained.

In operation, it is preferred that the system be calibrated prior to immersion of probe 10. A calibration for zero percent of the measured gases is accomplished by flow of carrier gas 20, usually argon, through mass spectrometer 64. To do so, valves 20 and 42 are caused to open thereby permitting turbo pump 66 to draw gas 20 through mass spectrometer 64 and out to the atmosphere through rotary vane, membrane, or scrolling pump 68. Excess gas also is allowed to flow through exhaust port 58.

In a next calibration step, a gas containing a known concentration of one of the test gases is drawn through one of the sources 22, 24, or 26 through solenoid valves 48, 46, or 44, respectively into mass spectrometer 64 with excess gas once again being evacuated through exhaust port 58 as needed. In a further step, which may take place simultaneously with calibration of the spectrometer, probe assembly 10 is purged with carrier gas 20 by opening solenoid valves 36 and 38.

Details of probe 10 are given more fully in the description of the '526 patent referred to above. During a purge cycle carrier gas 20 is purged through inflow ports 14 and 16 as well as outflow port 15. During testing after immersion of the probe in molten metal 17, carrier gas 20 is caused to flow through solenoid valve 36 and through outflow port 15 through lines illustrated on FIG. 1. After flowing through the molten metal and reaching equilibrium with gases in the molten metal, the gas to be tested is drawn through outlet port 16 and through lines 61 and 60, bypassing manifold valve assembly 40, into the mass spectrometer 64.

In each of these described test procedures an electronic signal is provided by mass spectrometer 64 to remote microprocessor 30. Microprocessor 30 is connected to the various solenoid valves, mass spectrometer 64 and various pumps used to operate the system by known electronic connections. Also illustrated by phantom lines is an enclosure 76. Enclosure 76 is preferably cooled by an inert coolant such as compressed air.

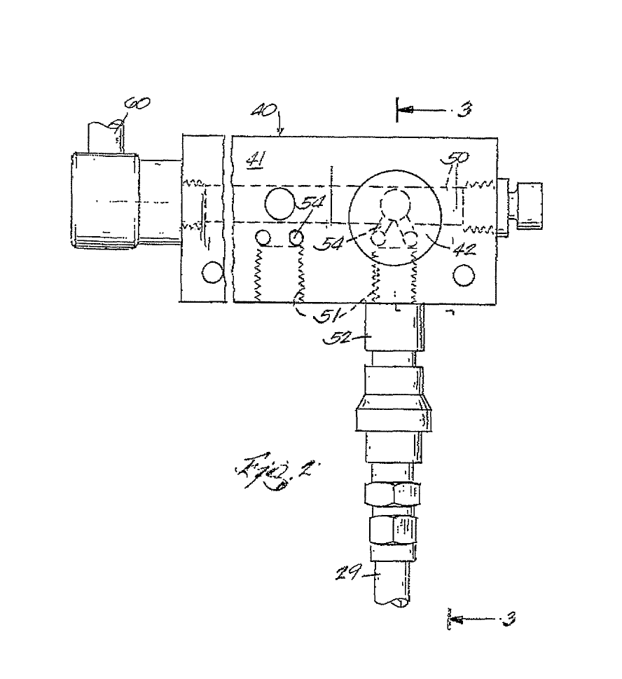

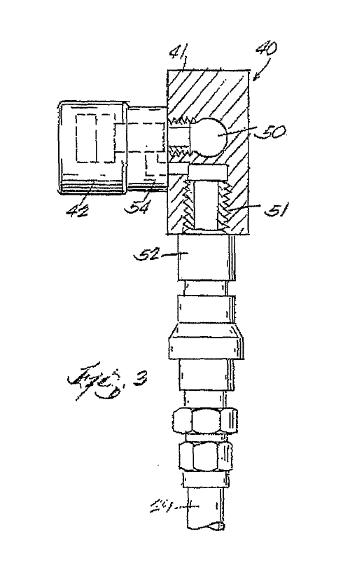

Referring to FIGS. 2 and 3, manifold 40 is shown in greater detail. Manifold 40 is provided with a central fluid flow channel 50, which may be continuously purged with carrier gas 20. Threaded sockets 51 receive conventional fittings 52 to connect fluid flow line 29 to manifold 40. In the illustrated embodiment, solenoid operated valves 42 are threaded into a channel open to central fluid flow channel 50. An interior channel 54 enables flow of the gases from conduit 29 into the solenoid actuated valve 42. It will be understood that appropriate O-rings and the like are utilized to provide a leak-proof channel.

The illustrated arrangement has been found to minimize residual gases that would interfere with successive use of various valves 42, 44, 46, and 48. It will be appreciated that since the calibration and purging gases can flow through manifold 40 to mass spectrometer 64 and since there is a bypass connection between probe 10 and mass spectrometer 64 through solenoid 62, that the calibration of spectrometer 64 can occur simultaneously with various operations such as immersion in metal 17 of probe 10 and purging thereof with the carrier gas 20. Therefore efficiency and speed of the test procedures are thus greatly enhanced.

Referring now to FIG. 4, there is seen a further embodiment of the invention. In accordance with this embodiment a copper oxide furnace 70 and a desiccant 72 are employed. Also preferably added in accordance with this embodiment is a gas reservoir 80. The embodiment of FIG. 4 is useful in eliminating interference of carbon monoxide with the accurate measurement of nitrogen concentration. Such interference occurs because both CO and N₂ have an atomic mass unit of 28. To eliminate the interference, the CuO furnace converts CO into CO₂.

In practice, a measurement is made of the gas emanating from probe 10. In this measurement the peaks for atomic mass units 2 (H₂), 12 (C), 14 (N), 28 (CO and N₂), and 44 (CO₂) are monitored. This measurement gives accurate readings for H₂ and CO₂. Then, the gas, which may be stored in gas reservoir 80, is passed through the CuO furnace 70 wherein it is preferably heated to approximately 400 degrees Celsius. Thereafter mass spectrometer 64 is again used to determine the peaks at atomic mass units 14, 28, and 44 for gas that has passed through CuO furnace 70. The increase in mass unit 44 reading is indicative of the amount of CO present. The remaining peak at atomic mass unit 28 is accurately indicative of the concentration of nitrogen.

Down stream from copper oxide furnace 70 may be a desiccant 72. Desiccant 72 prevents deleterious introduction of water into mass spectrometer 64. The preferred desiccant is CaSO₄, commercially available under the trade name DRIERITE.

The preferred material for the gas reservoir shown in FIG. 4 is polyvinyl fluoride (PVF), commercially available under the trade name TEDLAR. Vacuum pumps 56 and appropriately positioned solenoid actuated valves are also utilized to control the flow of the gases. In practice, during the purge sequence for probe 10, the gas reservoir bag 80 is emptied using the vacuum pumps 56 and then sealed with a solenoid actuated valve to make it ready for sampling. Once the test commences, mass spectrometer 64 monitors the gas extracted from molten metal 17. When a steady state is achieved, the gas stream is diverted into gas reservoir bag 80 and collected. When the initial testing is complete, some of the gases retained in reservoir 80 are diverted through CuO furnace 70 and then analyzed using mass spectrometer 64.

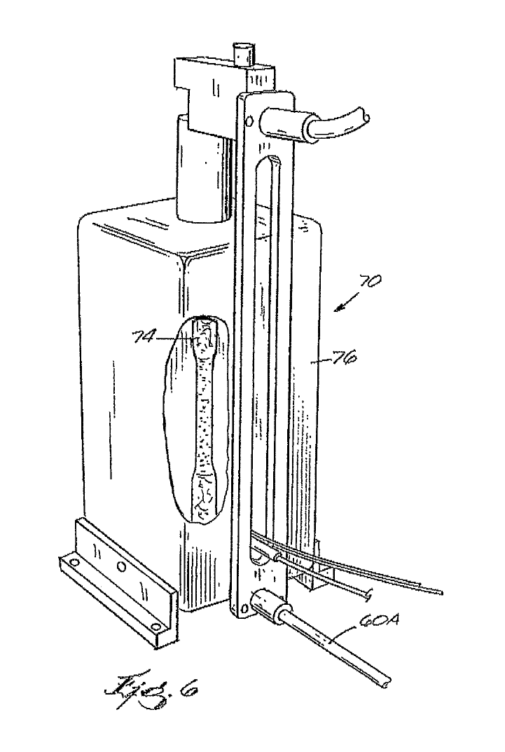

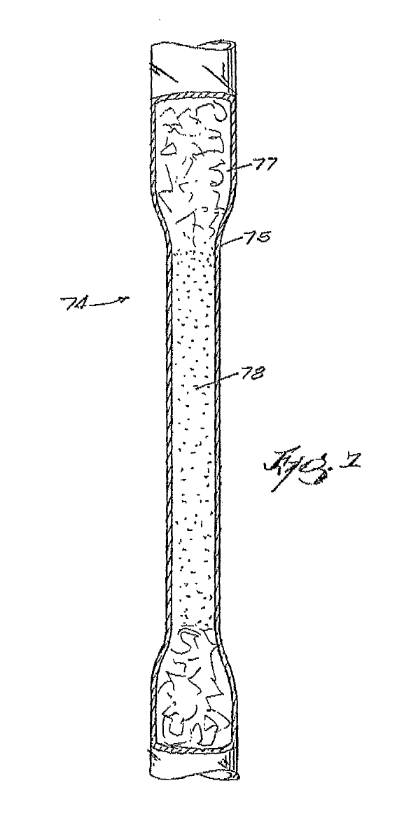

Referring to FIGS. 6 and 7, furnace 70 includes a treating chamber 74 that consists of an elongated tube 75 formed of a heat resistance material such as quartz. CuO material 78, as well as packing such as fibrous ceramic or glass material 77, are contained within tube 75 through which gases supplied through a conduit such as 60A are caused to flow.

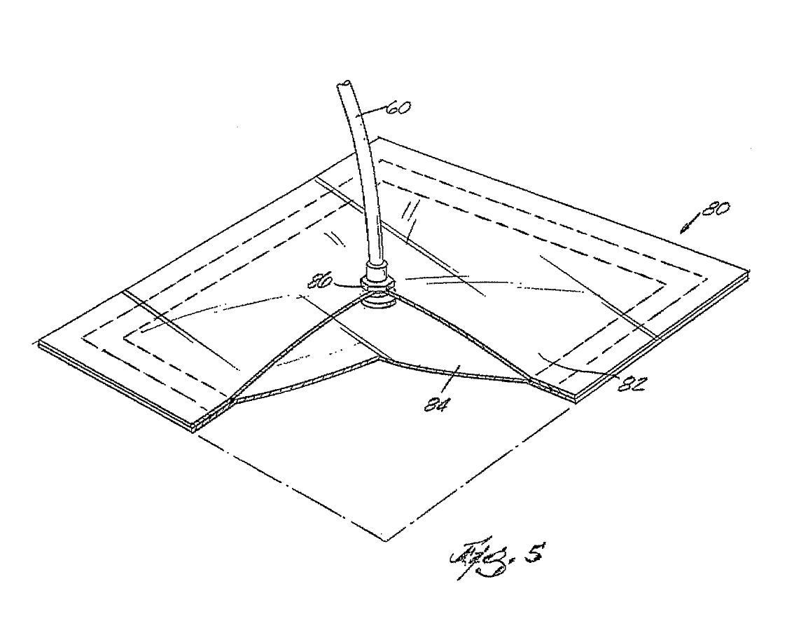

While the use of the gas reservoir bag 80 provides special advantages to the embodiment of FIG. 4, it is also useful for any testing of gases where more than a few seconds of test time is required. Suitable reservoir bags are commercially available from Cole-Parmer Corporation under the trade name TEDLAR. Gas reservoir bag 80 is shown in greater detail in FIG. 5. Bag 80 consists of a closed pocket formed by upper face 82 and lower face 84, which are bonded along their perimeter, for example by adhesives or heat sealing. A threaded fitting 86 is provided on one side to enable leak proof attachment of a fluid flow conduit such as conduit 60.

1. A method for determination of the concentration of gases dissolved in molten metal comprising:
providing a molten metal immersible probe, a gas analysis device, and a gas flow conduit interconnecting said probe and said gas analysis device;
immersing said probe in a molten metal;
introducing a stream of inert carrier gas into said metal through said probe;
recovering said carrier gas as said gas bubbles out of said metal;
using said analysis device to determine the content of at least one gas contained in the gas flowing out of said metal;
passing a second amount of said carrier gas recovered from said metal through an oxidizing medium, thereby converting carbon monoxide contained in said gas to carbon dioxide and converting hydrogen contained in said gas to water vapor;
testing the gas after passage thereof through said oxidizing medium, and comparing the readings obtained from analysis of said two gas streams to thereby accurately determine the concentration of nitrogen, hydrogen and carbon monoxide contained in said gas.

2. A method according to claim 1 wherein said second amount of said gas stream is temporarily stored in a reservoir prior to analysis thereof.

3. A method according to claim 1 wherein the gas stream passing out of said oxidizing medium is passed through a desiccant material to remove water vapor therefrom.

4. A method according to claim 3 wherein said desiccant material comprises CaSO₄.

5. A method according to claim 1 wherein said oxidizing medium comprises copper oxide heated in a copper oxide furnace.

6. A method according to claim 1 wherein said gas analysis device is a quadrupole mass spectrometer.

7. A method according to claim 1 wherein the concentrations of hydrogen, nitrogen and oxygen in the molten metal are determined.

8. A method for determination of the concentration of gases dissolved in molten metal comprising:
providing a molten metal immersible probe and a gas analysis device;
providing a plurality of sources of calibration gases containing known concentrations of the gases to be tested for;
providing a fluid flow conduit interconnecting each of said sources of calibration gases with a valve manifold assembly, at least one solenoid-controlled valve controlling the flow of each of said calibration gases into said manifold, a bypass gas flow conduit interconnecting said probe and said gas analysis device independently of said manifold;
introducing said plurality of calibration gases, sequentially, into said analysis device and analyzing said calibration gases to calibrate the analysis device;
immersing said probe in a molten metal;
introducing a stream of inert carrier gas into said metal through said probe;
recovering said carrier gas as said gas bubbles out of said metal and causing said gas to flow into said analysis device through said bypass gas flow conduit; and
using said analysis device to determine the content of at least one gas contained in the gas flowing out of said metal.

9. A method according to claim 8 wherein said probe is immersed in said molten metal during calibration of said analysis device.

10. A method according to claim 8 wherein said analysis device is a mass spectrometer.

11. Apparatus for determination of the concentration of gases dissolved in molten metal comprising:
a molten metal immersible probe and a gas analysis device;
a plurality of sources of calibration gases containing known concentrations of the gases to be tested for;
a fluid flow conduit interconnecting each of said sources of calibration gases with a valve manifold assembly;
at least one solenoid-controlled valve controlling the flow of each of said calibration gases into said manifold;
a fluid flow conduit interconnecting said manifold and said gas analysis device; and
a bypass gas flow conduit interconnecting said probe and said gas analysis device independently of said manifold.

12. Apparatus for determination of the concentration of gases dissolved in molten metal comprising:
a molten metal immersible probe;
a gas analysis device;
a first gas flow conduit interconnecting said probe and said gas analysis device;
an oxidizing medium;
a second gas flow conduit interconnecting said probe with said oxidizing medium and with said gas analysis device;
solenoid controlled valves for controlling gas flow through said first and second gas flow conduits; and
a microprocessor for controlling said valves and for comparing readings obtained from analysis of a first gas stream flowing directly to said analysis device from said probe with a second gas stream flowing to said analysis device through said oxidizing medium to thereby determine the concentration of nitrogen, hydrogen and carbon monoxide contained in said gases.

13. Apparatus for determination of the concentration of gases dissolved in molten metal comprising:
a molten metal immersible probe and a gas analysis device;
a first fluid flow conduit interconnecting said probe and said gas analysis device;
a solenoid-controlled valve controlling the flow of gases between said probe and said gas analysis device,
a gas storage reservoir;
a second fluid flow conduit interconnecting said probe with said gas reservoir and said gas reservoir with said gas analysis device; and
a solenoid-controlled valve controlling the flow of gases between said probe and said gas storage reservoir.

14. Apparatus according to claim 13 wherein said reservoir comprises a bag formed of polyvinyl fluoride.

15. A method for determination of the concentration of gases dissolved in molten metal comprising:
providing a molten metal immersible probe and a gas analysis device, a first fluid flow conduit interconnecting said probe and said gas analysis device and a solenoid-controlled valve controlling the flow of gases between said probe and said gas analysis device;
providing a gas storage reservoir, a second fluid flow conduit interconnecting said probe with said gas storage reservoir and said gas storage reservoir with said gas analysis device, and a solenoid-controlled valve controlling the flow of gases between said probe and said gas storage reservoir;
immersing said probe in molten metal and recovering gases therefrom;
storing a portion or said recovered gases in said reservoir;
subsequently causing recovered gases to flow from said storage reservoir to said analysis device; and
determining the concentration of at least one gas in the gases flowing from said gas storage reservoir.

16. A method according to claim 15 wherein said reservoir comprises a bag formed of polyvinyl fluoride.
